(19) United States
(12) Patent Application Publication
(10) Pub. No.: US 2007/0225589 A1
(43) Pub. Date: Sep. 27, 2007

(54) Single catheter diagnosis, navigation and treatment of arrhythmias

(76) Inventor: Raju R. Viswanathan, St. Louis, MO (US)

Correspondence Address: HARNESS, DICKEY, & PIERCE, P.L.C, 7700 BONHOMME, STE 400, ST. LOUIS, MO, 63105, US

(21) Appl. No.: 11/329,958

(22) Filed: Jan. 11, 2006

Related U.S. Application Data
(60) Provisional application No. 60/642,852, filed on Jan. 11, 2005.

(51) Int. Cl. A61B 5/05

(52) U.S. Cl. 600/407

(57) ABSTRACT

A method is provided for controlling an electrophysiological catheter in combination with a navigation system, an ECG recording system, and an algorithm for directing the movement of the catheter. The method comprises navigating the distal end of the catheter to sense intra-cardiac activation signals at a minimum number of locations on the wall of a subject body's heart, recording the local intra-cardiac signal data for the minimum number of locations, and determining the direction of propagation of the wave from with respect to time from the minimum number of location points using the algorithm. The method further comprises calculating a new location point in the direction of the source of the signal propagation wave front using the algorithm, for use with at least two of the prior locations for further evaluation of the wave front direction, iteratively repeating the step of determining the direction of propagation to obtain the earliest activation location of the wave front, and responsively navigating the distal tip of the catheter to the earliest activation location for providing medical treatment.

### CROSS-REFERENCE TO RELATED APPLICATIONS

This application claims the benefit of U.S. provisional patent application Ser. No. 60/642,582 filed Jan. 11, 2005, the entire disclosure of which is incorporated herein by reference.

### BACKGROUND OF THE INVENTION

Cardiac arrhythmias are a form of cardiac disease where the electrical activity of the heart is disrupted, often due to the takeover of signal generation by abnormal excitation nodes.

Cardiac arrhythmia may be treated through minimally invasive interventions such as catheter ablation, where catheters navigate a set of electrodes (often 3-8 electrodes) intravascularly into the relevant chambers of the heart, and monitor electrical signal activation times and propagation to thereby identify location points of focal arrhythmias, for example Supraventricular Tachycardia (e.g,. SVT). An electro-physiological study is performed to record the activation sequence at target locations of the heart, to determine the arrhythmia mechanism. Such mapping may then be used to identify location points within the heart that are part of the tachycardia or arrhythmia mechanism, but not part of the normal cardiac conduction system. Such location points are then rendered electrically inactive by ablating the point, typically by Radio Frequency ablation. Recent advancements have also resulted in automated remote navigation systems that can drive catheter placement with a great deal of precision, more specifically magnetic navigation systems.

### SUMMARY OF THE INVENTION

Embodiments of the systems and methods of the present invention advance the art of remote surgical navigation by combining diagnosis with navigation and therapy, using a minimal number of devices. In one embodiment of the present invention, a system is provided for treatment of arrhythmia that comprises an electrophysiological catheter having at least one electrode for sensing intra-cardiac wave front activation signals on a tissue surface, a navigation system for guiding the distal end of the catheter to a number of locations for sensing intra-cardiac activation signals along the wall of a subject body's heart, and an ECG recording system for recording the local intra-cardiac signal data for each of the locations. The system further comprises a computer for determining the direction of propagation of the wave front with respect to time from the intra-cardiac activation signals corresponding to the location points. From the direction of propagation of the wave front, the computer may calculate a new location point in the direction of the source of the wave front for advancing the catheter to, where intra-cardiac activation data may be used with at least two of the prior locations for further evaluation of the wave front direction.

In another aspect of the present invention, a method is provided for determining the movements of the catheter towards a focal arrhythmia for ablation. A method is provided in combination with a navigation system, a localization system, and an algorithm for directing the movement of the catheter. The method comprises navigating the distal end of the catheter to sense intra-cardiac activation signals at a number of locations on the wall of a subject body's heart, recording the local intra-cardiac signal data for the minimum number of locations, and determining the direction of propagation of the wave from with respect to time from the location points using the algorithm. The method further comprises calculating a new location point in the direction of the source of the wave front using the algorithm, for use with at least two of the prior locations for further evaluation of the wave front direction, iteratively repeating the step of determining the direction of propagation to obtain the earliest activation location of the wave front, and responsively navigating the distal tip of the catheter to the earliest activation location for providing medical treatment.

### BRIEF DESCRIPTION OF THE DRAWINGS

FIG. 1 is a cut away view of a heart showing a re-entry circuit within the right atria and a possible target area for ablation to render the re-entry mechanism inactive;

FIG. 2 is an illustration of the difference in phase of ECG signals at points p₁ and p₂; and

FIG. 3 is an illustration of a number of location points of intra-cardiac activity having phase signal differences, and a corresponding direction of wave front propagation.

### DETAILED DESCRIPTION OF THE INVENTION

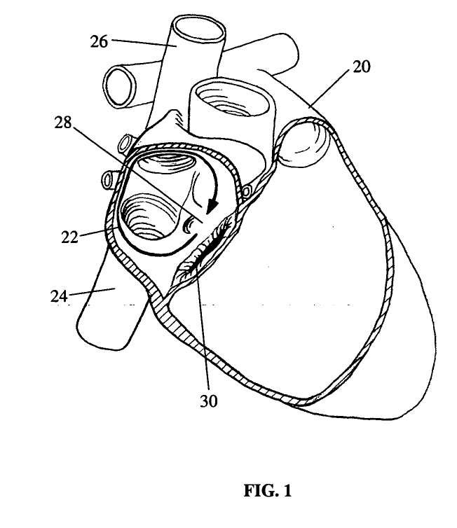

In a preferred embodiment of the present invention, a system and method are provided for measuring and recording various points in a region of a patient's heart for mapping electrophysiological activity of the tissue, and for determining a target location for catheter ablation to correct an arrhythmia mechanism. The arrhythmia mechanism of an atrioventricular re-entry tachycardias may be established where an electrical wave front occurs within the heart that generates a re-entry circuit. In the cutaway of a heart 20 in FIG. 1, an example of a re-entry circuit is generally shown as a circular electrical pathway 22 within the right atrium around the inferior vena cava 24 and superior vena cava 26. A possible target 28 for atrial ablation, for example, could be near the isthmus between the tricuspid valve 30 and the inferior vena cava 24. Ablation would render the location electrically inactive, and would interrupt the electrical pathway of the re-entry circuit. Atrioventricular Nodal Re-entry Tachycardia is another arrhythmia mechanism that is established where both a fast and slow conduction pathways into the atrioventricular node exist. Atrial flutter and Focal Atrial Tachycardia are yet further re-entry mechanisms in which the passage of the activation wave front around the atrium establishes re-polarization of the ventricle before the wave front completes one circuit. The present invention provides a method for evaluating such various arrhythmia mechanisms and determining the source or focal point of the arrhythmia to be treated. The method described herein involves using a single catheter to measure and record intracardiac electrical activity in a small local region, identify the direction of signal propagation of the wave front of intracardiac activity from these measurements, and navigate the catheter in the appropriate direction towards the source of the wave front. Once identified, such electrical signal sources that are not part of the normal cardiac conduction system can be removed by catheter ablation techniques such as Radio Frequency (RF) ablation, where electrical energy is delivered through the tip electrode of the catheter in order to locally destroy abnormal tissue.

In one preferred embodiment in accordance with the present invention, a system for treatment of arrhythmia is provided that comprises a catheter having at least one electrode for sensing intra-cardiac wave front activation signals on a tissue surface, and a navigation system for guiding the distal end of the catheter to a minimum number of locations to sense intra-cardiac activation signals along the endocardial wall of a subject's heart. Navigation of the catheter may be performed by a magnetic navigation system or any other navigation system suitable for guiding a catheter within a subject body. An ElectroPhysiology ECG recording system is used for recording the location the local intra-cardiac electrical signal data corresponding to the minimum number of locations. In one preferred embodiment, a localization system is used to record catheter tip location data together with intra-cardiac electrical signal data. The system may also include a fluoroscopic imaging system for obtaining images and location points of the catheter within the body during the surgical procedure. In an alternate embodiment, fluoro-localization is used to record three dimensional catheter tip location data by manually marking on corresponding points in at least two fluoro images.

In a preferred embodiment, the catheter may be a magnetically navigable catheter, which may be advanced through the vasculature in a selected direction by pushing the proximal end of the catheter, and by deflecting the distal end of the catheter by an applied magnetic field to gain entry to a vessel branch. The distal end of the catheter may comprise a radio-opaque material useful for viewing in an X-ray or fluoroscopic imaging system, and one or more magnetic elements which can be deflected to align with an applied magnetic field external to the subject body of a patient. One such navigation system, for example, is the Stereotaxis Niobe™ magnetic navigation system, which can apply an external magnetic field of about 0.08 Tesla within the subject in any direction in order to suitably orient or steer the catheter. In alternate embodiments, other actuation schemes such as mechanical, electrostrictive, hydraulic or other methods could be used to steer or deflect the catheter in order to navigate it.

The system further comprises a computer for determining the regional direction of propagation of the wave front from the intra-cardiac signal data corresponding to the location points. By determining the direction of propagation of the wave front, the computer calculates a new location for advancing the catheter in the direction of the source of the wave front, where intra-cardiac signal data may be used with at least two of the prior locations for further evaluation and adjustment of the estimated wave front direction. The computer may execute an algorithm for iteratively repeating the above progression to determine the earliest activation location or source of the wave front, and responsively navigating the distal tip of the catheter to the earliest activation location for medical treatment.

The preferred embodiment further comprises a method for determining the point of earliest activation of a local wave front associated with focal atrial tachycardia. The method includes the step of determining the direction of propagation of the wave front from an analysis of signal delays or signal arrival times in the intra-cardiac signal data corresponding to the location points. By determining the direction of the propagation of the wave front, the method calculates a new location for advancing the catheter in the direction of the source of the wave front, where intra-cardiac signal data may be used with at least two of the prior locations for further evaluation or estimation of the wave front propagation direction. The method repeats the iterative progression to determine the earliest activation location of the wave front and to responsively navigate the distal tip of the catheter to the earliest intra-cardiac activation location for medical treatment.

The system and method may automatically determine the location of a focal point of arrhythmia or atrial tachycardia re-entry mechanism where unpolarized intra-cardiac activation is initiated, and may automatically advance the catheter to the location for ablation treatment. The method may also be used to perform an electrophysiological study for generating an electro-anatomical map of the heart tissue. Such atrial tachycardia re-entry or other cardiac arrhythmia mechanisms are established by lines of conduction that can be visualized using mapping systems that can characterize and predict focal points. The advantages of the methods used in the present invention to evaluate measured local intracardiac activation data and to responsively determine the propagation of the wave front of intracardiac activation for moving the mapping/ablation catheter to a desired location for ablation will become apparent from the following detailed description of the method.

Step 1

The catheter tip is positioned at three locations on the wall of the heart chamber and the electrical signals recorded at each of these locations. The locations are preferably mutually separated by separations in the range 5 mm-20 mm and more preferably in the range 5 mm-15 mm. An ECG system (ideally outputting data to the navigation system) records local intracardiac signal data at each of these locations p₁, p₂, and p₃. At p₁, the ECG data is recorded for about 3-20 cycles to determine the periodicity T of the signal. The position \vec{X}₁ can be determined, for example by fluoro-localization.

Step 2

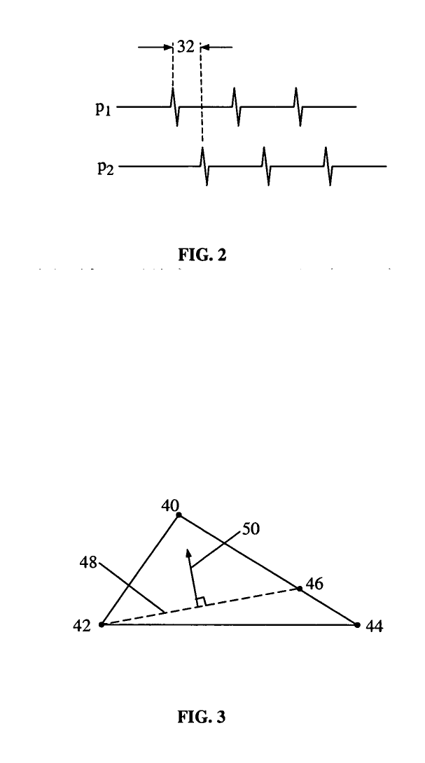

The catheter is moved to location p₂, and its position \vec{X}₂ is determined, for example by fluoro-localization. The electrical signal is recorded and its phase difference with respect to the signal at p₁, is measured. FIG. 2 illustrates the phase difference 32 of signals at p₁, and p₂. If the signal (peak) at p₂ is measured at time τ, Δ₂=(τ−NT) where N is the largest integer such that Δ₂ is positive. If Δ₂>T/2, define a′=−(T−Δ₂ ), else define a′=Δ₂; a′ is the phase difference at p₂.

The catheter is then moved to location p₃, and its position \vec{X}p₃ is determined, for example by fluro-localization. The electrical signal is recorded, and its phase difference b′ is determined.

Step 3

The points are relabeled as needed such that p₁, is the point of earliest activation, i.e., a′ and b′ (phase differences at the other 2 points with respect to p₁,) are both positive, and are hereinafter referred to as a and b instead of a′ and b′.

The triangle formed by points p₁, (40), p₂, (42), and p₃ (44) is shown in FIG. 3. The three triangle points p₁, (t=0), p₂, (t=a), and p₃ (t=b) all have associated time or propagation delays relative to the other points. This is a small (local) triangle, and therefore the time (propagation) delays within this triangle may be linearly interpolated with little error. Isochrones (contours of equal propagation time) within this triangle represent the local wave front; the direction of propagation is normal to this wave front. Referring to FIG. 3, where b>a (no loss of generality), the isochrone passing through point \vec{X}₂ (42) is the dotted line 48, intersecting edge x₁−x₃ of the triangle at a point \vec{X}₀ (46), such that

$${\vec{x}}_{0} = {\vec{x}}_{1} + \frac{a}{b} ( {\vec{x}}_{3} - {\vec{x}}_{1} )$$

(since propagation delays are linearly interpolated within the triangle). The vector \vec{l}=(\vec{x}₀−\vec{x}₂) is therefore along the isochronal direction \vec{n} (or at equal time propagation). Since the propagation direction 50 must be perpendicular to this,

$$\begin{matrix}\vec{n} \cdot \vec{ℓ} = o , or \vec{n} \cdot ( {\vec{x}}_{0} - {\vec{x}}_{2} ) = 0. \\ Therefore , & ( 1 ) \\ \vec{n} = α {\vec{u}}_{1} + β {\vec{u}}_{2} \\ where & ( 2 ) \\ {\vec{u}}_{1} = \frac{( { \vec{x}}_{1} - { \vec{x}}_{2} )}{\vert {\vec{x}}_{1} - {\vec{x}}_{2} \vert} \\ and {\vec{u}}_{2} = \frac{( {\vec{x}}_{3} - {\vec{x}}_{2} )}{\vert {\vec{x}}_{3} - {\vec{x}}_{2} \vert} \\ \vec{n} is a unit vector , so we have & ( 3 ) \\ {α}^{2} + {β}^{2} + 2 α β cos ϑ = 1 & ( 4 )\end{matrix}$$

where cos θ=\vec{u}₁·\vec{u}₂.

From equations (1) and (2):

$$\vec{n} \cdot ( {\vec{x}}_{o} - {\vec{x}}_{2} ) = 0 or \\ ( α {\vec{u}}_{1} + β {u}_{2} ) \cdot [ b ( {\vec{x}}_{1} - {\vec{x}}_{2} ) + a ( {\vec{x}}_{3} - {\vec{x}}_{1} ) ] = 0 \\ or \\ α [ b {\vec{u}}_{1} \cdot ( {\vec{x}}_{1} - {\vec{x}}_{2} ) + a {\vec{u}}_{1} \cdot ( {\vec{x}}_{3} - {\vec{x}}_{1} ) ] + β [ b {\vec{u}}_{2} \cdot ( {\vec{x}}_{1} - {\vec{x}}_{2} ) + a {\vec{u}}_{2} \cdot ( {\vec{x}}_{3} - {\vec{x}}_{1} ) ] = 0 \qquad ( 5 )$$

Equations (4) and (5), can be solved for α and β, and thus n can be determined (pick the sign of \vec{n} such that \vec{n} points towards \vec{x}₁, or such that n has positive dot product with the vector (x₁−(x₀+x₂)/2)).
Step 4

Once n (the local reverse propagation direction) is determined, starting at \vec{x}₁ a new point \vec{y}′₁=A\vec{n}+\vec{x}₁ is defined where A is a step size in the range 5 mm-20 mm. \vec{y}′₁ is defined as a new target for the catheter; because the wall surface is curved, target navigation of the catheter (with suitable control actuations applied) will actually take the tip to a location \vec{y}₁. A new triangle O₂ is formed by the points \vec{y}₁ and the 2 points (from triangle O₁) that are closest to it. The process is iteratively repeated to get a new local propagation direction in triangle O₂, as long as the activation time at point \vec{y}₁ is earlier than that of the other 2 points in O₂. If the activation time at \vec{y}₁ is later than that of at least one of the other 2 points, a reduced step is taken:

$$Define {\vec{𝓏}}_{1}^{′} = \frac{A}{2} \vec{n} + {\vec{x}}_{1}$$

and navigate the catheter to a (real) wall location \vec{z}₁, etc.
Step 5

In a relatively small number of steps/iteration, the focal point of the arrhythmia may thus be found and the catheter will have been placed there. Ablative therapy may be performed to eliminate the source of the arrhythmia.

It is worth noting that these methods may be generalized to multi-focal arrhythmias by looking for double periodicities and other signal features, such that multiple isochrones may be tracked locally to arrive at multiple foci. Likewise more than one catheter may be used in combination for diagnosis and navigation. The remote navigation system could be used with a localization system with location feedback, or with a registered pre-operative or other anatomical data. In the latter case, a suitably modified stepping point \vec{y}₁ etc. may be directly defined on the (curved) heart surfaces so that a stepped path is defined on the curved surface, minimizing the need for repeated fluoro localization. Although fluoro-localization has been described in the example detailed above, in the case where real-time location data is available from a device localization system, fluro-localization is not needed, again minimizing the need for repeated user interaction. In an alternate embodiment, catheter tip location could be estimated or evaluated from a knowledge of actuation control variables from the navigation system and a computational device model that predicts tip location based on the actuation controls. Varying levels of automation thus are possible depending a system integration and availability of anatomical and/or catheter location data.

What is claimed is:

1. A system for controlling an electrophysiological catheter to detect the location of arrhythmias, the system comprising:
a catheter having at least one electrode for sensing intra-cardiac wave front activation signals on a tissue surface;
a navigation system for guiding the distal end of the catheter to selected locations on the wall of a subject's heart;
a processor for determining the direction of propagation of the intra-cardiac wave front with respect to time from electric signals sensed by the at least one electrode on the catheter at a plurality of locations, and determining a new location at which to sense the intra-cardiac wave front based upon the determined direction of propagation of the intra-cardiac wave front.

2. The system according to claim 2 wherein the processor further controls the navigation system to navigate the catheter to the newly determined location.

3. The system according to claim 1 further comprising an ECG recording system for recording the local intra-cardiac signal data for each of the plurality of locations.

4. The system according to claim 1 wherein the processor determines the new location based upon electric signals at three locations.

5. The system according to claim 1 wherein the processor uses signal data from the new location, and signal data from at least two previous locations to determine another new location.

6. The system according to claim 1 wherein the processor implements algorithm for evaluating the intra-cardiac signals from the locations and iteratively determining the direction of propagation of the wave front to obtain the earliest activation location of the wave front.

7. The system according to claim 2 wherein the processor determines the direction of the propagation of the wave front from a contour of equal time propagation calculated from the phase difference between the locations, and a vector normal to the contour.

8. The system according to claim 1 wherein the distal end of the catheter is automatically guided by the navigation system to the determined new location without input further by the user.

9. The system according to claim 1 wherein the distal end of the catheter is automatically guided by the navigation system to the determined new location after a user input.

10. The system of claim 1 wherein the processor repeats the steps of determining the direction of propagation of the intra-cardiac wave front with respect to time from electric signals sensed by the at least one electrode on the catheter, determining a new location at which to sense the intra-cardiac wave front based upon the determined direction, and causing the navigation system to navigate the catheter to the newly determined location, until the earliest activation location is determined.

11. The system of claim 10 wherein the catheter comprises an ablation means for ablating tissue the earliest activation location to render the location electrically inactive.

12. The system of claim 11, wherein the system comprises only one electrophysiology catheter.

13. The system of claim 11, wherein the system includes a second catheter that is manually navigable to obtain additional intracardiac ECG data.

14. A method for controlling at least one electrophysiology catheter for detecting of arrhythmia using a navigation system, the method comprising:
navigating the distal end of the electrophysiology catheter to sense intra-cardiac electrical activation signals from at least three locations on the endocardial wall of a subject's heart;
recording local intra-cardiac signal data from each of the at least three locations; and
determining a new location to sense the intra-cardiac electrical activation signals based upon the local intra-cardiac signal data from at least three locations, and
navigating the distal end of the catheter to the new location.

15. The method according to claim 14 wherein determining a new location based upon the local intra-cardiac signal data from at least three locations comprises determining the direction of propagation of a signal propagation front from the local intra-cardiac signal data from the at least three locations and determining the new location point in the direction of the source of the wave front.

16. The method according to claim 15 further comprising recording local intra-cardiac signal data at the new location, and determining a further new location based upon the local intra-cardiac signal data at the new location and local intra-cardiac signal data previously determined.

17. The method according to claim 14 further comprising recording local intra-cardiac signal data at the new location, and determining a further new location based upon the local intra-cardiac signal data at the new location and local intra-cardiac signal data previously determined.

18. The method according to claim 14 further comprising iteratively repeating the steps of recording local intra-cardiac signal data at a current location, determining a new location based the local intra-cardiac signal at the current location and at least one previous location, and moving the catheter to the new location, until the earliest activation location is located.

19. The method according to claim 16 further comprising using the catheter to ablate tissue at the earliest activation location.

20. The method of claim 19 wherein the steps are performed with only one catheter.

21. The method of claim 19 wherein the steps are performed with at least two catheters.

22. The method of claim 15, wherein the direction of the propagation of the wave front from a contour of equal-time propagation is calculated from the signal phase difference between the at least three locations as a vector normal to the contour.

23. The method of claim 14 wherein the step of navigating the distal end of the catheter to various locations to sense local intra-cardiac signals, and the step of navigating the catheter to the calculated new location point is automatically performed by the navigation system.

26. The method of claim 18 wherein the step of determining a new location based the local intra-cardiac signal at the current location and at least one previous location comprises using a progression of successive triangles, where each triangle comprises at least two points from a prior triangle and the new location.

27. A method for controlling an electrophysiological catheter with a computerized navigation system, the method comprising:
(a) navigating the distal end of the catheter to sense intra-cardiac activation signals at at least three locations on the wall of a subject's heart;
(b) recording the local intra-cardiac signal data at each of the at least three locations;
(c) determining the direction of propagation of the signal propagation front from the local intra-cardiac signal data;
(d) determining a new location point in the direction of the source of the wave front;
(e) navigating the distal end of the catheter to the new location;
(f) recording the local intra-cardiac activation signals at the new location; and iteratively repeating steps (c) through (d) to determine the earliest activation location of the wave front.

28. The method according to claim 27 further comprising ablating the earliest activation location point to render the location electrically inactive.

29. The method according to claim 27 further comprising ablating the earliest activation location point to render the location electrically inactive, using the same catheter used to sense the intra-cardiac activation signals.

30. The method of claim 27 wherein the direction of propagation of the signal propagation wave front is computed from a contour of equal time propagation, that is determined from the phase difference between the locations, as a vector normal to the contour.

31. The method of claim 27, wherein the distal end of the catheter is automatically guided by the navigation system to various locations to sense local intra-cardiac signals, and the catheter is automatically advanced to the new location for determining the next earliest activation location of the wave front.
